(19) United States
(12) Patent Application Publication
(10) Pub. No.: US 2014/0350367 A1
(43) Pub. Date: Nov. 27, 2014

(54) PEAK FLOW METER, BREATHALYZER, AND PULSE OXIMETER FOR USE WITH A MOBILE DEVICE

(71) Applicant: GestIn Time, Inc., Palo Alto, CA (US)

(72) Inventor: Suresh SUBRAMANIAM, Palo Alto, CA (US)

(73) Assignee: GestIn Time, Inc., Palo Alto, CA (US)

(21) Appl. No.: 13/899,559

(22) Filed: May 21, 2013

(51) Int. Cl. A61B 5/00; A61B 5/1455; A61B 5/08

(52) U.S. Cl. CPC A61B 5/0022; A61B 5/0803; A61B 5/082; A61B 5/14552
USPC 600/340; 600/538; 600/532

(57) ABSTRACT

A peak flow meter, breathalyzer, and pulse oximeter for use with a mobile device, such as a smartphone, are disclosed. In one embodiment, the peak flow meter receives air flow from the patient, converts it into an electrical signal using a pressure transducer, and transmits the signal to a mobile device over an interface. In another embodiment, the microphone of the mobile device is used to directly receive the airflow, and the mobile device converts the airflow into an electrical signal indicating the peak flow. In another embodiment, a breathalyzer receives air flow from a patient, converts it into an electrical signal using a sensor, and transmits the signal to a mobile device over an interface. In another embodiment, a pulse oximeter measures oxygen saturation in a patient's blood and transmits an electrical signal for that measurement to a mobile device over an interface.

### TECHNICAL FIELD

Embodiments of an improved peak flow meter, breathalyzer, and pulse oximeter for use with a mobile device is disclosed.

### BACKGROUND OF THE INVENTION

Peak flow meters are well-known medical devices. A peak flow meter can measure a patient's peak expiratory flow (typically measured in liters per minute), which relates to the patient's ability to breathe out air. Peak flow meters are often used by patients to monitor asthma or other respiratory conditions.

The prior art includes peak flow meters that are portable. A patient exhales into a tube or small opening in the device. The device measures the peak expiratory flow using a pressure transducer and displays the result with a mechanical gauge or on an electrical display. A low reading can indicate an asthma attack or other condition.

Prior art peak flow meters provide a result for the patient to view. However, there is no way for the data to be automatically communicated to a doctor or medical professional. There also is no way for the data to be automatically recorded for future reference. This is a significant limitation of the prior art, since trends in peak expiratory flow can indicate if a patient's condition is worsening and in need of medical attention.

What is needed is an improved peak flow meter that is capable of communicating data to a doctor or medical professional automatically and/or to upload the data to a server. What is further needed is a mechanism for recording data over time, displaying trends, and generating reports.

Breathalyzers and pulse oximeters also are known in the art and have the same limitations discussed above for prior art peak flow meters. What is needed is an improved breathalyzer and improved pulse oximeter that are capable of communicating data to a doctor or medical professional automatically and/or to upload the data to a server. What is further needed is a mechanism for recording data over time, displaying trends, and generating reports.

### SUMMARY OF THE INVENTION

The aforementioned problem and needs are addressed through embodiments of a peak flow meter for use with a mobile device, such as a smartphone. In one embodiment, the peak flow meter receives air flow from the patient, converts it into an electrical signal using a pressure transducer, and transmits the signal to a mobile device over an interface. In another embodiment, the microphone of the mobile device is used to directly receive the airflow, and the mobile device converts the airflow into an electrical signal indicating the peak flow. Embodiments also are disclosed for a breathalyzer and pulse oximeter for use with a mobile device.

### BRIEF DESCRIPTION OF THE DRAWINGS

FIG. 1 depicts a prior art peak flow meter.

FIG. 2 depicts an embodiment of a peak flow meter for use with a mobile device.

FIG. 3 depicts a mobile device for use as a peak flow meter.

FIG. 4 depicts another embodiment of a peak flow meter for use with a mobile device

FIG. 5 depicts a method for determining peak flow using the microphone of a mobile device.

FIG. 6 depicts the components of a mobile device and a system for facilitating communication between the mobile device and a server and additional computing device.

FIG. 7 depicts an exemplary report of peak flow data generated by a server or mobile device.

FIG. 8 depicts an embodiment of a medical device used in conjunction with a mobile device.

FIG. 9 depicts components of a mobile device.

FIG. 10 depicts an embodiment of a medical device used in conjunction with a web server.

FIG. 11 depicts an embodiment of a report generated using data stored in a data store.

### DETAILED DESCRIPTION OF THE PREFERRED EMBODIMENTS

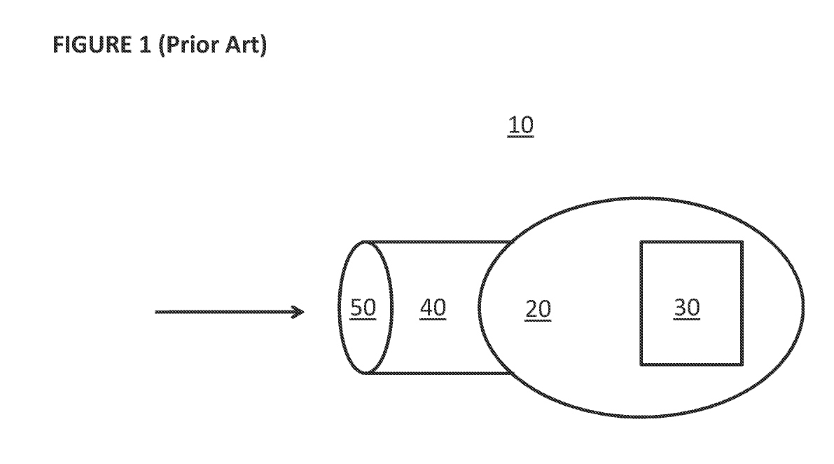

A prior art peak flow meter 10 is shown in FIG. 1. Peak flow meter 10 comprises shaft 40, opening 50, pressure transducer 20, and display 30. The patient places shaft 40 in his or her mouth and blows into opening 50, in the direction shown by the arrow. Pressure transducer 20 converts pressure from the airflow into an electrical signal, which is then shown on display 30, either as numerical data or through another interface such as a graphical representation of a gauge.

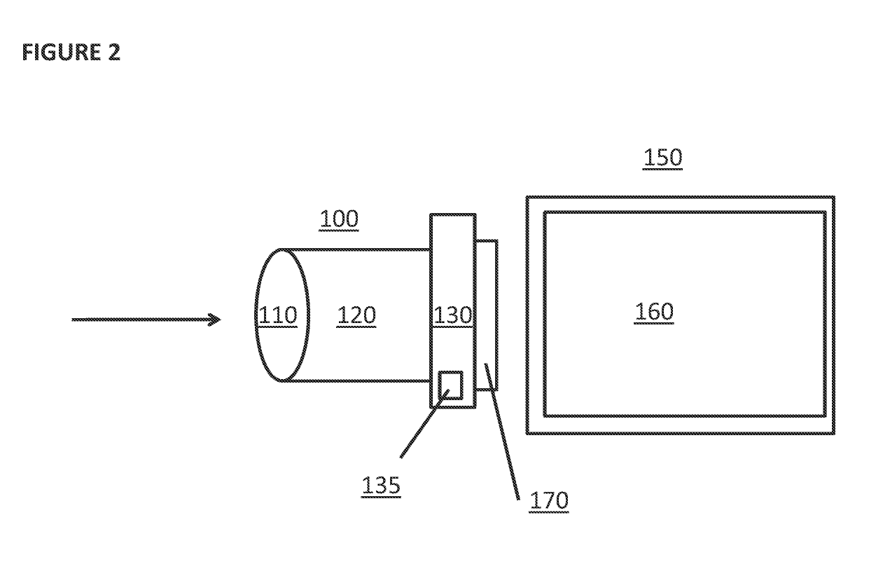

An embodiment of an improved peak flow meter 100 is shown in FIG. 2. Peak flow meter 100 includes shaft 120 and opening 110 (which are similar to shaft 40 and opening 50 in FIG. 1), the patient places shaft 120 in his or her mouth and blows into opening 110, in the direction shown by the arrow. Pressure transducer 130 converts pressure from the airflow into an electrical signal. The electrical signal is transmitted over input/output interface 170 to mobile device 150.

Mobile device 150 optionally can be a smartphone, such as an Apple iPhone® or Samsung Galaxy® device. However, mobile device 150 can be any mobile computing device that includes a processor, memory, and input/output port for connecting to input/output interface 70. Mobile device 150 also includes display 160, which optionally can be a touchscreen.

Input/output interface 170 optionally can be an Apple 30-pin, Apple Lightning, USB, Micro USB, or other connector. Input/output interface 170 optionally can also be a wireless interface, such as a Bluetooth interface or Wifi (e.g., 802.11) interface, in which case mobile device 150 would communicate with input/output interface 170 using a similar wireless interface on mobile device 150 instead of a physical port.

Mobile device 150 receives the electrical signal from pressure transducer 170 over input/output interface 170 and displays the result on display 160 using a graphical or numerical display. For example, the display 160 could simply show the reading of the peak pressure flow (e.g., “590 L/min”).

Peak flow meter 100 optionally can receive power from mobile device 150 over input/output interface 170. In one embodiment, peak flow meter 100 includes no battery and can receive all power necessary for its operation over input/output interface 170. In another embodiment, peak flow meter 100 includes a rechargeable battery 135 that is charged with the power received over input/output interface 170.

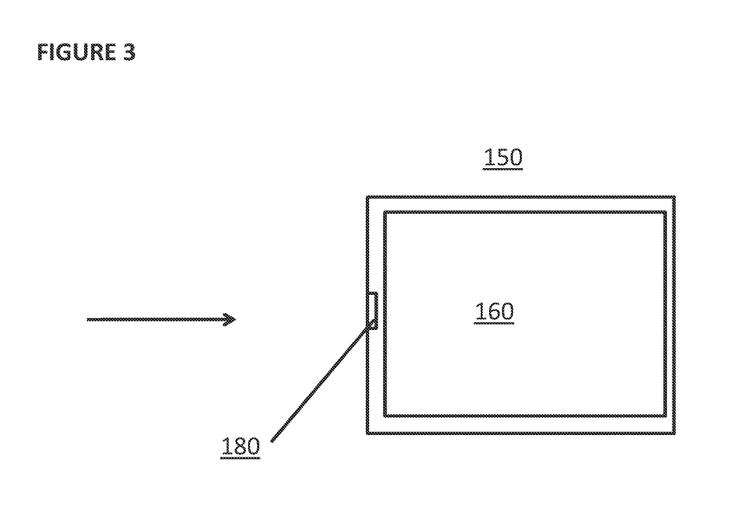

Another embodiment is shown in FIG. 3. Mobile device 150 comprises display 160 and microphone 180. Microphone 180, by its very nature, is a pressure sensor. The patient can breathe into microphone 180. Mobile device 150 will correlate the readings from microphone 180 with peak pressure flow. It will then display the peak pressure flow on display 160 as described previously for FIG. 2.

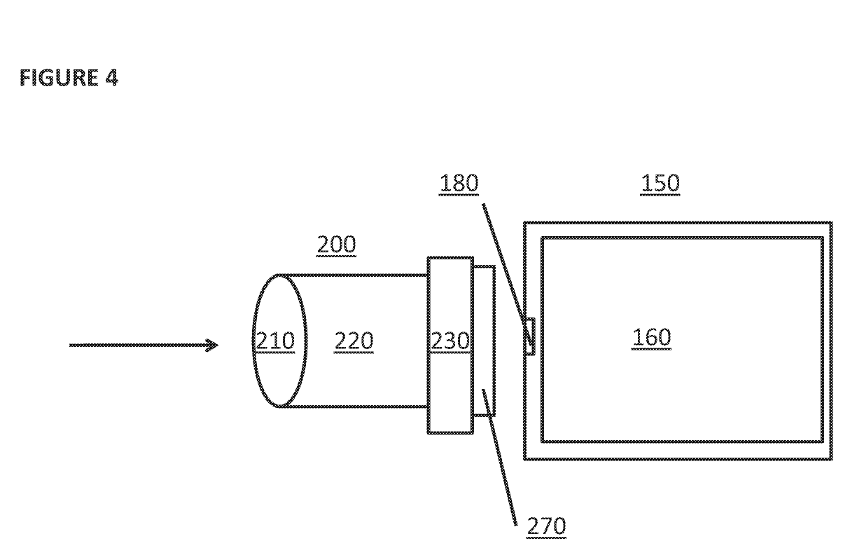

FIG. 4 depicts another embodiment. Here, device 200 comprises shaft 220 and opening 210, and the patient breathes into the opening 210 in the same manner described for previous embodiments. However, device 200 does not contain a pressure transducer. Instead, enclosure 230 directs the airflow received through opening 210 toward microphone 180. Device 200 includes connector 270, which can physically connect to a port on mobile device 150. However, connector 270 does not convey any electrical signals to mobile device 150 and serves only as a mechanical mounting for device 200. Connector 270 optionally can comprise an Apple 30-pin, Apple Lightning, USB, Micro USB, or other connector. Microphone 180 acts as a pressure sensor to determine peak flow as was the case with FIG. 3.

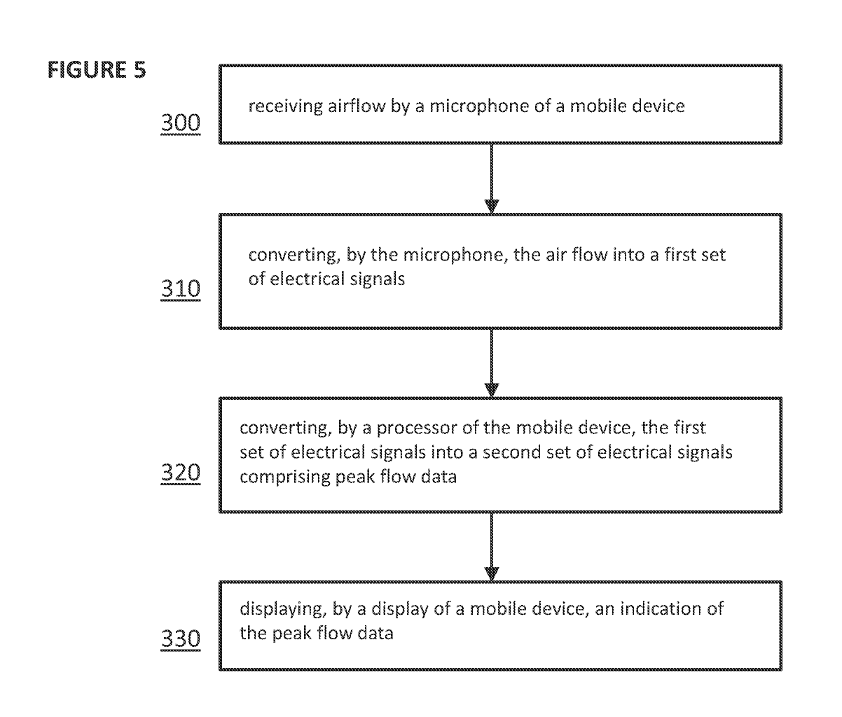

FIG. 5 depicts a method performed in conjunction with the apparatus of FIG. 3 or the apparatus of FIG. 4. The method is performed using mobile device 150, and comprises the steps of receiving, by a microphone of the mobile device, air flow (step 300), converting, by the microphone, the air flow into a first set of electrical signals (step 310), converting, by a processor of the mobile device 150, the first set of electrical signals into a second set of electrical signals, wherein the second set of electrical signals comprises peak flow data (step 320), and displaying, by a display 160 of the mobile device 150, an indication of the peak flow data (step 330).

The embodiments described thus have involved improved peak flow meters. However, some of the same principles can be applied to other medical devices, such as breathalyzers and pulse oximeters.

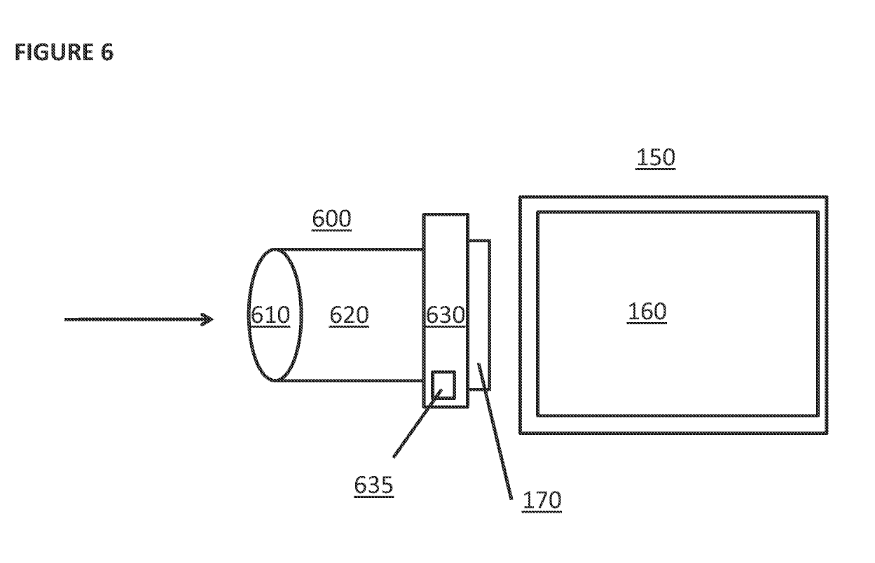

Prior art breathalyzers are known. They are devices that estimate the amount of alcohol in a person's body based on a sample of their breath. A breathalyzer converts air flow into an electronic signal representing the estimated blood alcohol content in the person's blood. FIG. 6 depicts a variation of FIG. 2. Breathalyzer 600 includes shaft 620 and opening 610. The user places shaft 620 in his or her mouth and blows into opening 610, in the direction shown by the arrow. Sensor 630 converts airflow into an electrical signal representing blood alcohol content. The electrical signal is transmitted over input/output interface 170 to mobile device 150. Input/output interface 170 and mobile device 150 are the same as the ones described previously with reference to FIG. 2.

Mobile device 150 receives the electrical signal from sensor 630 over input/output interface 170 and displays the result on display 160 using a graphical or numerical display. For example, the display 160 could simply show the reading of estimated blood alcohol content (e.g., “0.08 grams of alcohol/210 liters breath”).

Breathalyzer 600 optionally can receive power from mobile device 150 over input/output interface 170. In one embodiment, breathalyzer 600 includes no battery and can receive all power necessary for its operation over input/output interface 170. In another embodiment, breathalyzer 600 includes a rechargeable battery 635 that is charged with the power received over input/output interface 170.

Similarly, pulse oximeters are known in the prior art. Pulse oximeters indirectly monitor the oxygen saturation of a patient's blood by estimating the percentage of arterial hemoglobin in the oxyhemoglobun configuration. Pulse oximeters typically emit light at different wavelengths through the user's fingernail and measure the amount of each type of light that is absorbed by the user. The amount of oxygen in the blood affects the absorption of the different types of light. The pulse oximeter generates an electronic signal representing the percentage of arterial hemoglobin in the oxyhemoglobun configuration.

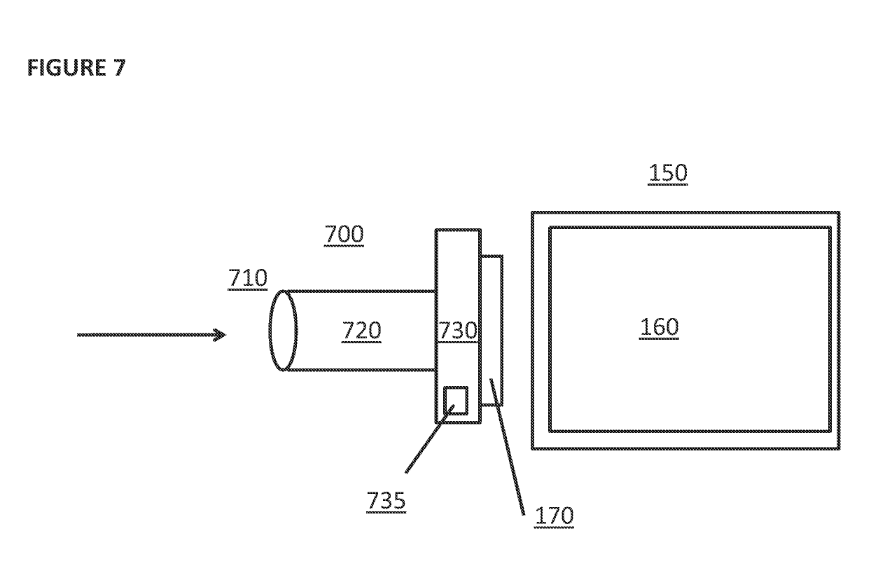

FIG. 7 depicts a variation of FIG. 2. Breathalyzer 700 includes shaft 720 and opening 710. The user places his or her finger into opening 710, in the direction shown by the arrow. Device 730 converts light absorption readings into an electrical signal representing oxygen saturation The electrical signal is transmitted over input/output interface 170 to mobile device 150. Input/output interface 170 and mobile device 150 are the same as the ones described previously with reference to FIG. 2.

Mobile device 150 receives the electrical signal from device 770 over input/output interface 170 and displays the result on display 160 using a graphical or numerical display. For example, the display 160 could simply show the reading of estimated blood alcohol content (e.g., “98%”).

Pulse oximeter 700 optionally can receive power from mobile device 150 over input/output interface 170. In one embodiment, breathalyzer 700 includes no battery and can receive all power necessary for its operation over input/output interface 170. In another embodiment, breathalyzer 700 includes a rechargeable battery 735 that is charged with the power received over input/output interface 170.

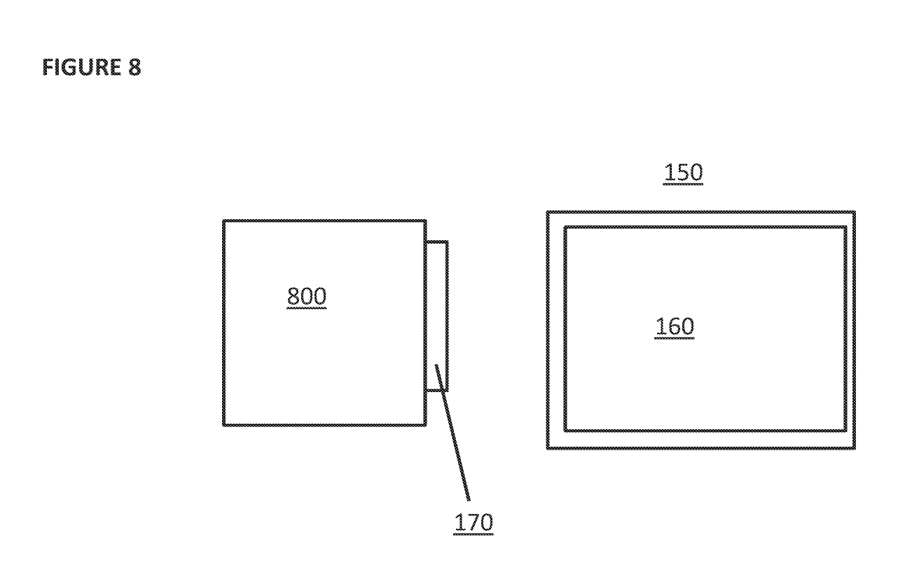

FIG. 8 depicts a more general variation of FIG. 2. Medical device 800 comprises input/output interface 170 for communicating with mobile device 150. Input/output interface 170 and mobile device 150 are the same as the ones described previously with reference to FIG. 2. Medical device 800 optionally can be a scale for determining a patient's weight, a sphygmomanometer for determining a patient's blood pressure, a glucometer for determining the blood sugar level of a patient's blood, or any other device that measures a patient's characteristics and generates results in the form of digital data.

Mobile device 150 receives the electrical signal from medical device 800 over input/output interface 170 and displays the result on display 160 using a graphical or numerical display. For example, the display 160 could simply show the reading of the patient's weight, blood pressure, or blood sugar level.

Medical device 800 optionally can receive power from mobile device 150 over input/output interface 170. In one embodiment, medical device 800 includes no battery and can receive all power necessary for its operation over input/output interface 170. In another embodiment, medical device 800 includes a rechargeable battery 735 that is charged with the power received over input/output interface 170.

In yet another embodiment, medical device 800 can provide multiple functions for a patient. For example, it can comprise a combination of two or more of the following systems describe previously: a peak flow meter, breathalyzer, pulse oximeter, scale, sphygmomanometer, glucometer, or any other device that measures a patient's characteristics and generates results in the form of digital data. This embodiment would have the benefit of integrated medical functions that are convenient to use for a patient.

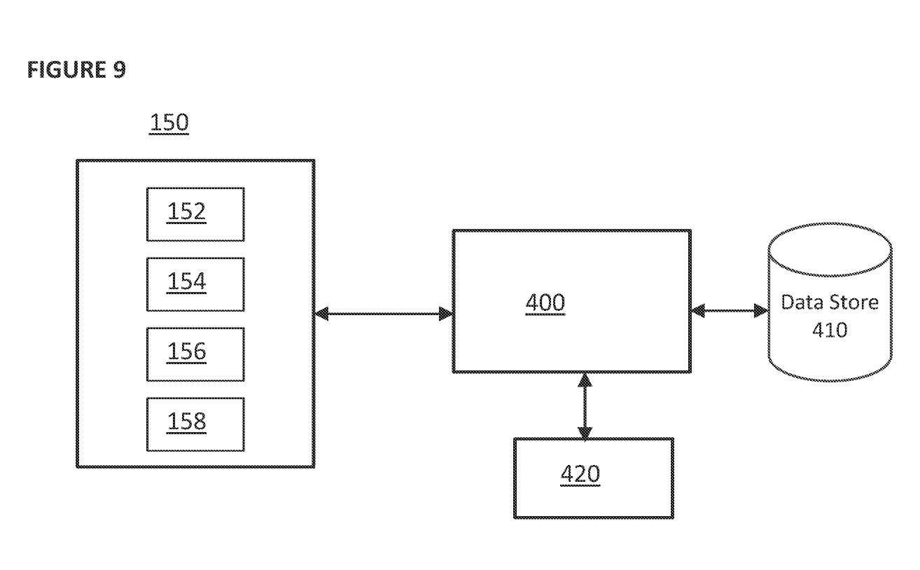

FIG. 9 depicts typical components of mobile device 150 described previously with reference to FIGS. 1-8. Mobile device 150 comprises processor 152, memory 154, data store 156, and network interface 158.

Data store 156 optionally can be a relational database for storing data records, such as a MySQL database, that is stored in non-volatile storage such as a hard disk drive or flash memory drive. Mobile device 150 optionally can communicates with server 400 over a network using network interface 158. The network optionally can be the Internet and can be hardwired, wireless, or some combination of the two. In one embodiment, network interface 158 is a wireless interface that utilizes 3G, 4G, or other cell phone wireless protocols, or uses a WiFi (802.11) protocol.

Server 400 comprises a datastore 410. Data store 410 optionally can be a relational database for storing data records, such as a MySQL database, that is stored in non-volatile storage such as a hard disk drive or flash memory drive.

Server 400 also can communicate with computing device 420 over a network. Computing device 420 can be a desktop, notebook, server, mobile phone, tablet, game console, or any other type of device with a processor, memory, and network interface. In this embodiment, computing device 420 is operated by a physician.

During operation of the embodiments of FIGS. 1-8, mobile device 150 records peak flow data in data store 156. Optionally, the data is sent automatically to server 400, where the data is stored in data store 410. Data store 156 optionally includes a table for the patient who operates mobile device 150. That table can be updated with data as it is transmitted from mobile device 150 to server 400. The data optionally can be recorded with data and time information so that trend data can be generated.

Optionally, the data can be sent by server 400 to computing device 420. Server 400 optionally can be configured to send an email, SMS or MMS message, or to place a phone call to computing device 420 if the data received from mobile device 150 satisfies predetermined criteria. For example, if the peak flow data received is below 500 L/minute, server 400 can be configured to send an email, SMS or MMS message, or to place a phone call to computing device 420 to indicate to the user of computing device 420 (who might be a physician or a relative of the owner of mobile device 150) that a troubling reading was received from mobile device 150.

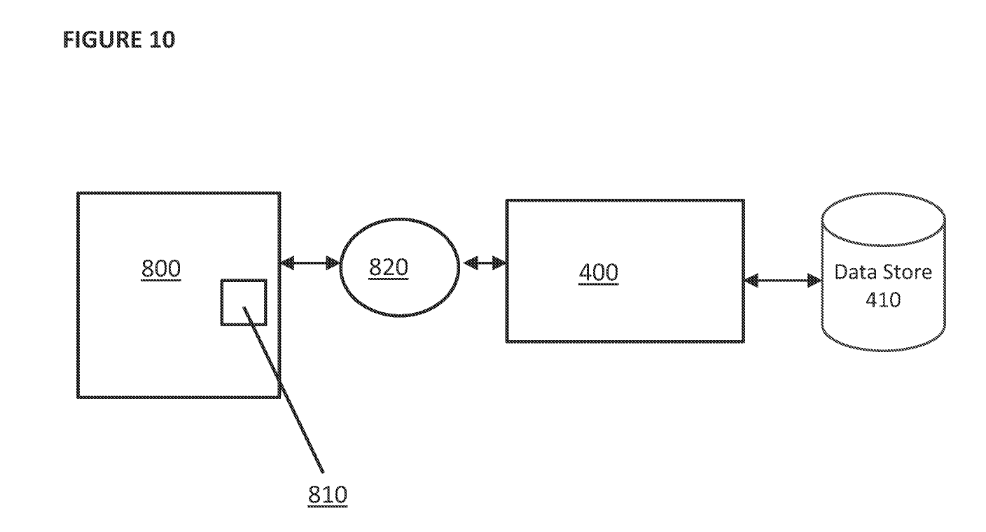

FIG. 10 depicts an embodiment where medical device 800 communicates directly with server 400 without the use of an intermediary mobile device. Medical device 800 comprises wireless interface 810. Wireless interface 810 comprises a wireless transceiver for communicating with network 820. Network 820 can comprise a 3G, 4G, or other cellular broadband network. Medical device 800 can communicate with server 400 over network 820 using known protocols, such as HTTP and other web protocols. Medical device 800 and server 400 otherwise can operate in the same manner as previously described.

In another embodiment, medical device 800 of FIGS. 9 and 10 can provide multiple functions for a patient. For example, it can comprise a combination of two or more of the following systems describe previously: a peak flow meter, breathalyzer, pulse oximeter, scale, sphygmomanometer, glucometer, or any other device that measures a patient's characteristics and generates results in the form of digital data. This embodiment would have the benefit of integrated medical functions that are convenient to use for a patient.

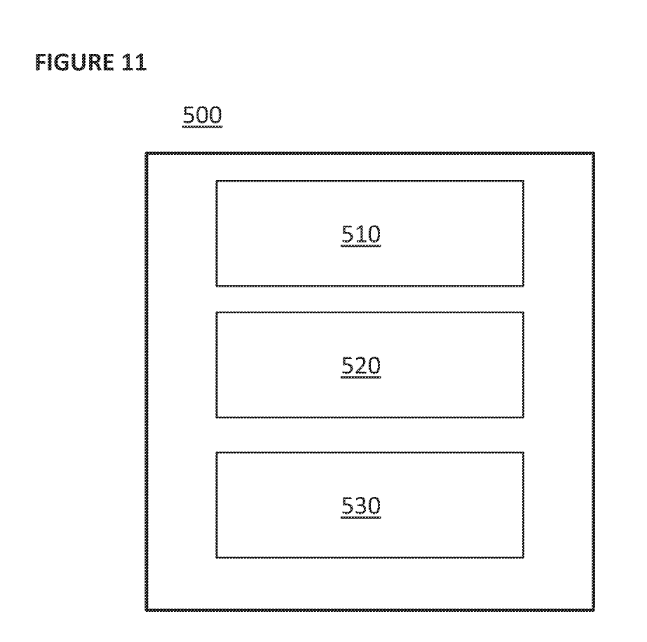

With reference now to FIG. 11, server 400 can generate a report 500 using data that is stored in data store 410 that is transmitted to mobile device 150 and/or computing device 420. In the alternative, report 500 can instead be generated locally by mobile device 150 using data stored in data store 156.

Report 500 optionally can comprise a first section 510 containing one or more graphs that track peak flow data over time. For example, first section 510 can contain graphs showing daily readings of peak flow data, average peak flow over certain time intervals, comparison of received peak flow data against the ideal data of a healthy patient, etc. Second section 520 optionally can include general assessments of trends of peak flow data (e.g., “Your average readings were higher this week than last week.). Third section 530 optionally can display messages from a physician or pharmacy (e.g., “Please schedule your next appointment soon.).

References to the present invention herein are not intended to limit the scope of any claim or claim term, but instead merely make reference to one or more features that may be covered by one or more of the claims. Materials, processes and numerical examples described above are exemplary only, and should not be deemed to limit the claims. It should be noted that, as used herein, the terms “over” and “on” both inclusively include “directly on” (no intermediate materials, elements or space disposed there between) and “indirectly on” (intermediate materials, elements or space disposed there between). Likewise, the term “adjacent” includes “directly adjacent” (no intermediate materials, elements or space disposed there between) and “indirectly adjacent” (intermediate materials, elements or space disposed there between). For example, forming an element “over a substrate” can include forming the element directly on the substrate with no intermediate materials/elements there between, as well as forming the element indirectly on the substrate with one or more intermediate materials/elements there between.

What is claimed is:

1. A peak flow meter, comprising:
a shaft with an opening for receiving air flow;
a pressure transducer;
an interface for connecting to a mobile device and for sending data from the pressure transducer to the mobile device.

2. The peak flow meter of claim 1, wherein the mobile device is a smartphone.

3. The peak flow meter of claim 1, wherein the peak flow meter is configured to obtain power over the interface.

4. The peak flow meter of claim 3, wherein the peak flow meter does not contain a battery.

5. The peak flow meter of claim 3, wherein the peak flow meter comprises a rechargeable battery.

6. The peak flow meter of claim 1, wherein the interface is a 30-pin interface.

7. The peak flow meter of claim 1, wherein the interface is a USB interface.

8. The peak flow meter of claim 1, wherein the interface is a Bluetooth interface.

9. A breathalyzer, comprising:
a shaft with an opening for receiving air flow from a user;
a sensor; and
an interface for connecting to a mobile device and for sending data from the sensor to a mobile device, wherein the data comprises an estimate of the user's blood alcohol content.

10. The breathalyzer of claim 9, wherein the mobile device is a smartphone.

11. The breathalyzer of claim 9, wherein the breathalyzer is configured to obtain power over the interface.

12. The breathalyzer of claim 11, wherein the breathalyzer does not contain a battery.

13. The breathalyzer of claim 11, wherein the breathalyzer comprises a rechargeable battery.

14. The breathalyzer of claim 9, wherein the interface is a 30-pin interface.

15. The breathalyzer of claim 9, wherein the interface is a USB interface.

16. The breathalyzer of claim 9, wherein the interface is a Bluetooth interface.

17. A pulse oximeter, comprising:
a shaft with an opening for receiving a user's finger;
a light emitting and detecting device;
an interface for connecting to a mobile device and for sending data from the light emitting and detecting device to a mobile device, wherein the data comprises an estimate of oxygen saturation in the user's blood;

18. The peak flow meter of claim 17, wherein the mobile device is a smartphone.

19. The peak flow meter of claim 17, wherein the peak flow meter is configured to obtain power over the interface.

20. The peak flow meter of claim 19, wherein the peak flow meter does not contain a battery.

21. The peak flow meter of claim 19, wherein the peak flow meter comprises a rechargeable battery.

22. The peak flow meter of claim 17, wherein the interface is a 30-pin interface.

23. The peak flow meter of claim 17, wherein the interface is a USB interface.

24. The peak flow meter of claim 17, wherein the interface is a Bluetooth interface.

25. A method of measuring peak flow using a mobile device, wherein the mobile device comprises a display, processor, and a microphone, comprising the steps of:
receiving, by the microphone, air flow;
converting, by the microphone, the air flow into a first set of electrical signals;
converting, by the processor, the first set of electrical signals into a second set of electrical signals, wherein the second set of electrical signals comprises peak flow data; and
displaying, by the display, an indication of the peak flow data.

26. The method of claim 25, wherein the mobile device is a smartphone.

27. The method of claim 25, further comprising:
displaying, by the display, a graph depicting a plurality of readings over time of peak flow data.

28. The method of claim 27, further comprising:
transmitting, by a network interface, the peak flow data to a server; and
storing, by the server, the peak flow data in a database table associated with a patient.
